(19) United States
(12) Patent Application Publication
(10) Pub. No.: US 2025/0135203 A1
(43) Pub. Date: May 1, 2025

(54) METHOD AND APPARATUS FOR NEUROSTIMULATION PROGRAMMING USING THERAPEUTIC EFFECT THRESHOLD

(71) Applicant: Boston Scientific Neuromodulation Corporation, Valencia, CA (US)

(72) Inventors: Lisa Denise Moore, Glendale, CA (US); G. Karl Steinke, Valencia, CA (US)

(21) Appl. No.: 18/905,770

(22) Filed: Oct. 3, 2024

Related U.S. Application Data
(60) Provisional application No. 63/546,471, filed on Oct. 30, 2023.

(51) Int. Cl. A61N 1/36; A61N 1/372

(52) U.S. Cl. CPC A61N 1/36135; A61N 1/3615; A61N 1/37247; A61N 1/37264

(57) ABSTRACT

A neurostimulation system may program a stimulation device to control delivery of neurostimulation according to a set of stimulation parameters and include a stimulation programming circuit. The stimulation programming circuit may include a sensed signal input, threshold detection circuity, and parameter setting circuity. The sensed signal input may be configured to receive sensed information indicative of a therapeutic effect improving a condition being treated using the neurostimulation. The threshold detection circuity may be configured to determine a therapeutic effect threshold of an adjustable parameter using the sensed information. The therapeutic effect threshold can be a minimum value of the adjustable parameter for the delivery of the neurostimulation according to the set of stimulation parameters to produce the therapeutic effect. The parameter setting circuity may be configured to determine the set of stimulation parameters for producing the therapeutic effect using the therapeutic effect threshold of the adjustable parameter.

### CLAIM OF PRIORITY

This application claims the benefit of U.S. Provisional Application No. 63/546,471, filed on Oct. 30, 2023, which is hereby incorporated by reference in its entirety.

### TECHNICAL FIELD

This document relates generally to neurostimulation and more particularly to a neurostimulation system that can be programmed for various operations using a threshold (e.g., a minimum value) of a stimulation parameter for neurostimulation to produce a therapeutic effect.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. These stimulation parameters need to be set to proper values to therapeutically benefit the patient without causing unacceptable side effects. These values may need to be reevaluated and adjusted over time to maintain the efficacy and/or safety of the neurostimulation in treating the patient. How these values of stimulation parameter are evaluated and determined can substantially affect the efficiency in programming the implantable neurostimulation system to achieve its desirable level of efficacy and safety.

### SUMMARY

An example (e.g., “Example 1”) of a system for delivering neurostimulation to a patient using a stimulation device is provided. The system may include a programming control circuit and a stimulation programming circuit. The programming control circuit may be configured to generate information for programming the stimulation device to control the delivery of the neurostimulation according to a set of stimulation parameters. The stimulation programming circuit may be configured to determine the set of stimulation parameters. The stimulation programming circuit may include a sensed signal input, threshold detection circuity, and parameter setting circuity. The sensed signal input may be configured to receive sensed information indicative of a therapeutic effect improving a condition being treated using the neurostimulation. The threshold detection circuity may be configured to determine a therapeutic effect threshold of an adjustable parameter of the set of stimulation parameters using the sensed information. The therapeutic effect threshold can be a minimum value of the adjustable parameter for the delivery of the neurostimulation according to the set of stimulation parameters to produce the therapeutic effect. The parameter setting circuity may be configured to determine the set of stimulation parameters for producing the therapeutic effect using the therapeutic effect threshold of the adjustable parameter.

In Example 2, the subject matter of Example 1 may optionally be configured such that the parameter setting circuity is configured to determine the set of stimulation parameters for producing the therapeutic effect in the patient using a minimum effective amplitude of a stimulation amplitude of the set of stimulation parameters, and the threshold detection circuity is configured to determine the minimum effective amplitude using the sensed information. The minimum effective amplitude is a minimum value of the stimulation amplitude for the delivery of the neurostimulation according to the set of stimulation parameters to produce the therapeutic effect in the patient.

In Example 3, the subject matter of any one or any combination of Examples 1 and 2 may optionally be configured such that the threshold detection circuity is configured to determine the therapeutic effect threshold of the adjustable parameter using stored sensed information.

In Example 4, the subject matter of any one or any combination of Examples 1 and 2 may optionally be configured such that the threshold detection circuity is configured to determine the therapeutic effect threshold of the adjustable parameter using a physiological signal of the sensed information. The physiological signal is sensed while the neurostimulation is delivered to the patient with the value of the adjustable parameter swept across a test range.

In Example 5, the subject matter of Example 4 may optionally be configured such that the threshold detection circuity is configured to determine the therapeutic effect threshold of the adjustable parameter in real time using the physiological signal sensed from the patient in real time.

In Example 6, the subject matter of Example 4 may optionally be configured such that the threshold detection circuity is configured to measure a biomarker parameter from the physiological signal, compare the measured biomarker parameter to a sensed effect threshold, and identify the therapeutic effect threshold of the adjustable parameter from the test range. The therapeutic effect threshold is a minimum value at which the measured biomarker parameter reaches or exceeds the sensed effect threshold.

In Example 7, the subject matter of any one or any combination of Examples 1 to 6 may optionally be configured such that the set of stimulation parameters defines a pattern of neurostimulation including one or more stimulation waveforms and one or more stimulation fields.

In Example 8, the subject matter of any one or any combination of Examples 1 to 7 may optionally be configured such that the sensed signal input is configured to receive a neural signal including evoked resonant neural activity (ERNA).

In Example 9, the subject matter of any one or any combination of Examples 1 to 8 may optionally be configured such that the parameter setting circuity is configured to set a value range of the adjustable parameter having a lower bound and an upper bound. The lower bound is set based on the therapeutic effect threshold of the adjustable parameter.

In Example 10, the subject matter of Example 9 may optionally be configured such that the parameter setting circuity is configured to set the upper bound of the value range of the adjustable parameter based on a side effect of the neurostimulation in the patient.

In Example 11, the subject matter of Example 9 may optionally be configured such that the parameter setting circuity is configured to set the value range of the adjustable parameter for identifying an optimal value of the adjustable parameter in a stimulation setting for the patient and to set the lower bound automatically to the therapeutic effect threshold of the adjustable parameter multiplied by a factor.

In Example 12, the subject matter of Example 9 may optionally be configured to further include a user interface including a presentation device and a user input device, and such that the parameter setting circuity is configured to set the value range of the adjustable parameter for identifying an optimal value of the adjustable parameter in a stimulation setting for the patient, to present the therapeutic effect threshold of the adjustable parameter using the presentation device, and to allow the lower bound to be set using the user input device.

In Example 13, the subject matter of Example 12 may optionally be configured such that the stimulation programming circuit further includes a notification circuitry to detect a substantial change in the therapeutic effect threshold of the adjustable parameter and to present a notification using the presentation device in response to a detection of the substantial change.

In Example 14, the subject matter of any one or any combination of Examples 1 to 8 may optionally be configured such that the parameter setting circuity is configured to determine values of the adjustable parameter in a stimulation program including a stimulation ramp during which the value of the adjustable parameter changes in increments from or to a bound or turning point and to set the bound or turning point based on the therapeutic effect threshold of the adjustable parameter.

In Example 15, the subject matter of any one or any combination of Examples 1 to 14 may optionally be configured such that the threshold detection circuity is configured to determine the therapeutic effect threshold of the adjustable parameter over time.

An example (e.g., “Example 16”) of a method for delivering neurostimulation to a patient using a stimulation device is also provided. The method may include: receiving sensed information indicative of a therapeutic effect improving a condition being treated using the neurostimulation, determining a therapeutic effect threshold of an adjustable parameter of a set of stimulation parameters using the sensed information, determining the set of stimulation parameters for producing the therapeutic effect using the therapeutic effect threshold of the adjustable parameter, and programming the stimulation device to control the delivery of the neurostimulation according to the set of stimulation parameters. The therapeutic effect threshold may be a minimum value of the adjustable parameter for the delivery of the neurostimulation according to the set of stimulation parameters to produce the therapeutic effect.

In Example 17, the subject matter of Example 16 may optionally further include sensing a physiological signal from the patient in real time, the subject matter of receiving the sensed information as found in Example 16 may optionally include receiving the physiological signal in real time, and the subject matter of determining the therapeutic effect threshold of the adjustable parameter as found in Example 16 may optionally include determining the therapeutic effect threshold of the adjustable parameter in real time.

In Example 18, the subject matter of Example 16 may optionally further include receiving stored sensed information including at least one of a previously sensed physiological signal or information derived from the previously sensed physiological signal, and the subject matter of determining the therapeutic effect threshold of the adjustable parameter as found in Example 16 may optionally include determining the therapeutic effect threshold of the adjustable parameter using the received stored sensed information.

In Example 19, the physiological signal as found in any one or any combination of Examples 17 and 18 may optionally include a neural signal including evoked resonant neural activity (ERNA).

In Example 20, the subject matter of determining the therapeutic effect threshold of the adjustable parameter as found in any one or any combination of Examples 17 to 19 may optionally include measuring a biomarker parameter from the physiological signal sensed while delivering the neurostimulation to the patient with the value of the adjustable parameter swept across a test range, comparing the measured biomarker parameter to a sensed effect threshold, and identifying the therapeutic effect threshold of the adjustable parameter from the test range. The therapeutic effect threshold is a minimum value at which the measured biomarker parameter reaches or exceeds the sensed effect threshold.

In Example 21, the subject matter of determining the set of stimulation parameters as found in any one or any combination of Examples 16 to 20 may optionally include determining a value range of the adjustable parameter having a lower bound and an upper bound, including setting the lower bound based on the therapeutic effect threshold of the adjustable parameter.

In Example 22, the subject matter of setting the lower bound based on the therapeutic effect threshold of the adjustable parameter as found in Example 21 may optionally include presenting the therapeutic effect threshold using a user interface and allowing a user to set the lower bound using the user interface.

In Example 23, the subject matter of determining the set of stimulation parameters as found in any one or any combination of Examples 16 to 22 may optionally include: determining values of the adjustable parameter in a stimulation program including a stimulation ramp during which the value of the adjustable parameter changes in increments from or to a bound or turning point, and setting the bound or turning point based on the therapeutic effect threshold of the adjustable parameter.

In Example 24, the subject matter of any one or any combination of Examples 16 to 23 may optionally further include: monitoring the therapeutic effect threshold of the adjustable parameter over time, detecting an unanticipated significant change of the therapeutic effect threshold of the adjustable parameter, and producing a notification in response to a detection of the unanticipated significant change.

An example (e.g., “Example 25”) of a non-transitory computer-readable storage medium including instructions, which when executed by a system, cause the system to perform a method for delivering neurostimulation to a patient using a stimulation device is also provided. The method may include: receiving sensed information indicative of a therapeutic effect improving a condition being treated using the neurostimulation, determining a therapeutic effect threshold of an adjustable parameter of a set of stimulation parameters using the sensed information, determining the set of stimulation parameters for producing the therapeutic effect using the therapeutic effect threshold of the adjustable parameter, and programming the stimulation device to control the delivery of the neurostimulation according to the set of stimulation parameters. The therapeutic effect threshold may be a minimum value of the adjustable parameter for the delivery of the neurostimulation according to the set of stimulation parameters to produce the therapeutic effect.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a stimulation programming circuit of a programming device of a neurostimulation system, such as the programming device of FIG. 3 or FIG. 8. The stimulation programming circuit can be configured for determining a therapeutic effect threshold for a patient and determining stimulation parameters for the patient based on the therapeutic effect threshold.

FIG. 10 illustrates another embodiment of the stimulation programming circuit of FIG. 9.

FIG. 11 illustrates an embodiment of a method for setting stimulation parameters using a therapeutic effect threshold.

FIG. 12 illustrates an embodiment of a method for determining the therapeutic effect threshold.

FIG. 13 illustrates an embodiment of a method for setting a value range for an adjustable stimulation parameter using the therapeutic effect threshold.

FIG. 14 illustrates an embodiment of a method for setting a stimulation ramp for an adjustable stimulation parameter using the therapeutic effect threshold.

FIG. 15 illustrates an embodiment of a method for setting the stimulation parameters using the therapeutic effect threshold adjusted over time.

FIG. 16 illustrates an embodiment of a method for producing a notification based on a substantial change in the therapeutic effect threshold.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that determines a therapeutic effect threshold of a stimulation parameter for a patient and/or a specific neurostimulation setting and uses the therapeutic effect threshold in programming a stimulation device for delivering a neurostimulation therapy to the patient. In various embodiments, the neuromodulation system can include an implantable device configured to deliver neurostimulation (also referred to as neuromodulation) therapies, such as deep brain stimulation (DBS), spinal cord stimulation (SCS), peripheral nerve stimulation (PNS), and vagus nerve stimulation (VNS), and one or more external devices configured to program or adjust the implantable device for its operations and monitor the performance of the implantable device.

When programming neurostimulation for a patient, it is helpful to know the threshold of a stimulation parameter (e.g., a minimum amplitude) at which an intended therapeutic effect (clinical improvement) is observable from the patient. Some signals sensed in response to delivery of neurostimulation can indicate such a therapeutic effect threshold. For example, a neural signal can be sensed from the patient while neurostimulation is delivered to the patient, and the amplitude of the sensed neural signal can be detected as a biomarker of a therapeutic effect. The amplitude of the sensed neural signal can therefore be used to determine the therapeutic effect threshold. In various embodiments, changes in the amplitude of the patient's response to the neurostimulation as detected from one or more sensed signals can indicate therapeutic effects of the neurostimulation. The value or changes in the value of frequency characteristics of the patient's response to the neurostimulation as detected from one or more (same or different) sensed signals can also indicate therapeutic effects of the neurostimulation. In this document, a “therapeutic effect” of neurostimulation can include an improvement of a patient's condition that is treated using the neurostimulation. A “therapeutic effect threshold” can be a minimum value of a stimulation parameter needed for producing a minimum therapeutic effect. A “minimum therapeutic effect” can be a minimum level of an intended therapeutic effect that is detectable by a device and/or a person. A “sensed effect threshold” can be a measure of a sensed signal indicative of the intended therapeutic effect that corresponds to the minimum therapeutic effect. The therapeutic effect threshold can be determined as the value of the stimulation parameter at which the sensed effect threshold is reached, when the signal is sensed during delivery of neurostimulation using various (e.g., sweeping) values of that stimulation parameter. A “minimum” value when associated with a threshold or effect can be the lowest value measured at a particular time or time period and/or for a specific setting of the neurostimulation (e.g., within a specific value range of a stimulation parameter), rather than an “absolute” or ‘global” minimum value.

The present subject matter provides a system in which one or more signals indicative of one or more minimum therapeutic effects are sensed and used to determine values and/or value ranges of stimulation parameters during initial setting, adjustments, and/or titration of neurostimulation programs. In various embodiments, the system can detect changes in the sensed one or more signals and adjust one or more stimulation parameters automatically and timely, while the patient is in a clinic or at home. In performing such adjustments, the system can also consider other factors related to each intended therapeutic effect, such as medications. In various embodiments, the present subject matter can be applied to various neurostimulation therapies, including but not being limited to DBS, SCS, PNJS, and VNS. In addition to automatically adjusting settings of neurostimulation, the system can present information generated using the sensed one or more signals, such as changes of therapeutic effects indicated by the sensed one or more signals, to users (e.g., clinicians programming the system for treating the patient) and/or the patient.

The one or more signals sensed according to the present subject matter can include any one or more physiological signals indicative of the one or more minimum therapeutic effects. The one or more signals can each include spontaneous and/or evoked activities indicative of one or more therapeutic effects. Examples of such one or more signals include neural signals. A specific example of such neural signal is a neural signal including evoked resonant neural activity (ERNA). The stimulation parameters for which the therapeutic effect threshold is used can include, but is not limited to, an amplitude (e.g., pulse amplitude in a pattern of neurostimulation pulses) and a frequency (or rate, e.g., pulse rate being the frequency of pulses in the pattern of neurostimulation pulses). While amplitude is discussed as a specific example in this document, the present subject matter can be applied to adjust any stimulation parameter having an impact on a therapeutic effect. In various embodiments, a neurostimulation program can have multiple intended therapeutic effects, which correspond to multiple therapeutic effect thresholds of one or more stimulation parameters. A set of such multiple therapeutic effect thresholds (which can be referred to as a “fingerprint”) can be established and saved for use in automatic adjustments of the neurostimulation program (e.g., one or more of its stimulation parameters) inside and/or outside a clinic.

A stimulation amplitude (e.g., a pulse amplitude) is discussed as an example, rather than a restriction, of a stimulation parameter in various embodiments of the present subject matter. The stimulation amplitude can have a “minimum effective amplitude” being its therapeutic effect threshold. A stimulation amplitude with a minimum effective amplitude is discussed as an example of, rather than a restriction to, stimulation parameters whose therapeutic effect thresholds can be determined and used according to the present subject matter.

In this document, unless noted otherwise, a “patient” includes a person receiving treatment delivered from, and/or being monitored and/or evaluated using, a neurostimulation system. A “user” includes a physician, other caregiver who examines, monitors, and/or treats the patient using the neurostimulation system, or other person who participates in the examination, monitoring, and/or treatment of the patient using the neurostimulation system (e.g., a technically trained representative, a field clinical engineer, a clinical researcher, or a field specialist from the manufacturer of the neurostimulation system).

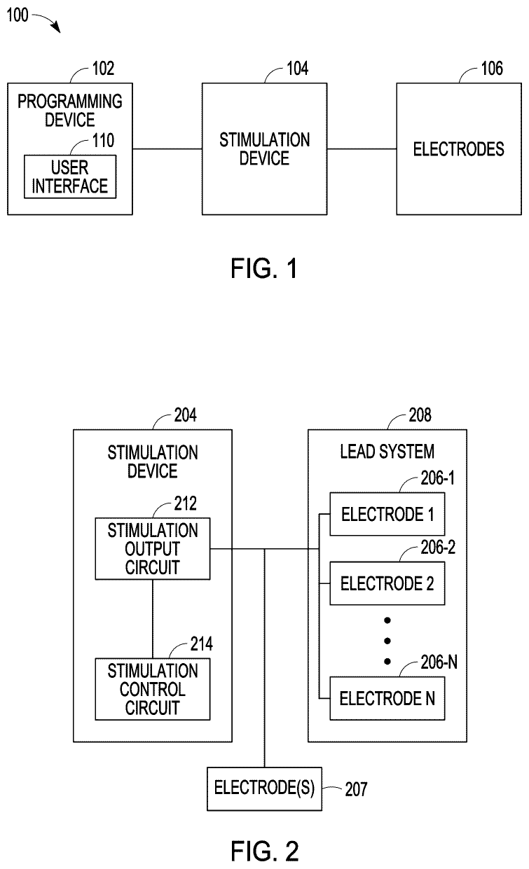

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In this document, a “user” includes a physician or other clinician or caregiver who examiners and/or treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. Such DBS configuration includes various features that may simplify the task of the user in programming stimulation device 104 for delivering DBS to the patient, such as the features discussed in this document.

FIG. 2 illustrates an embodiment of a stimulation device 204 and a lead system 208, such as may be implemented in neurostimulation system 100. Stimulation device 204 represents an embodiment of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

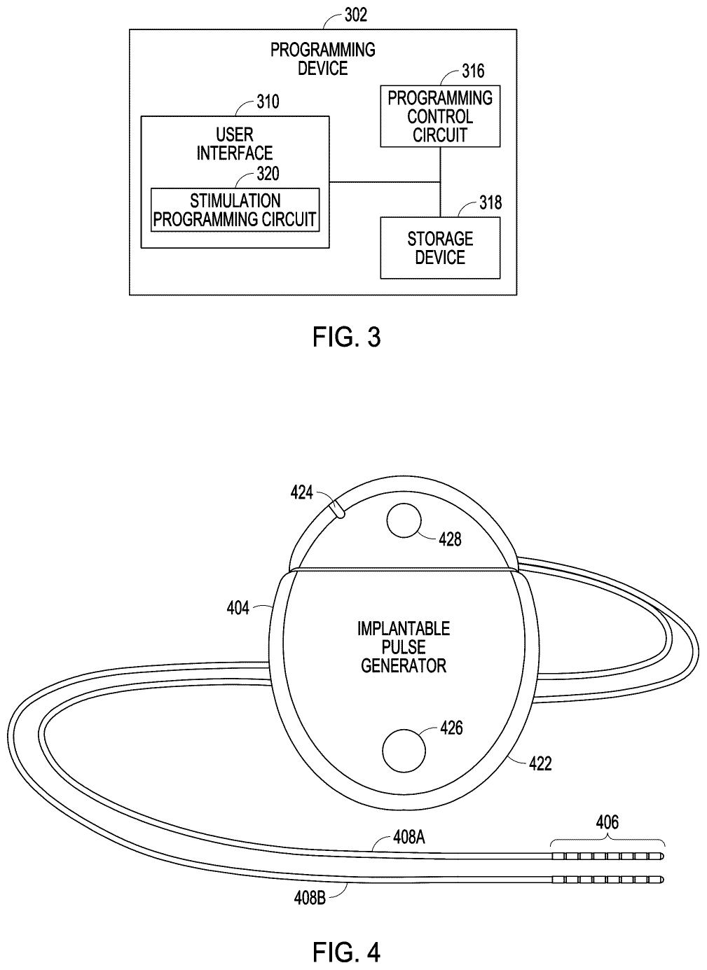

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions supporting the programming of stimulation devices, such as stimulation device 104 including its various embodiments as discussed in this document, including determining a therapeutic effect threshold for a patient and using the therapeutic effect threshold in programming stimulation parameters controlling delivery of neurostimulation to the patient.

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

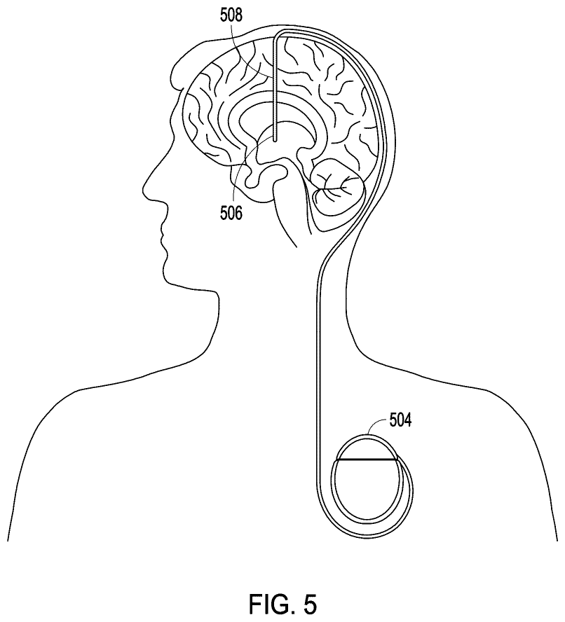

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrical contacts or electrodes 406 for contacting a tissue site targeted for electrical neurostimulation. As illustrated in FIG. 1, leads 408A and 408B each include 8 electrodes 406 at the distal end. The number and arrangement of leads 408A and 408B and electrodes 406 as shown in FIG. 1 are only an example, and other numbers and arrangements are possible. In various embodiments, the electrodes are ring electrodes. The implantable leads and electrodes may be configured by shape and size to provide electrical neurostimulation energy to a neuronal target included in the subject's brain, or configured to provide electrical neurostimulation energy to a nerve cell target included in the subject's spinal cord.

FIG. 5 illustrates an embodiment of an IPG 504 and an implantable lead system 508 arranged to provide neurostimulation to a patient. An example of IPG 504 includes IPG 404. An example of lead system 508 includes one or more of leads 408A and 408B. In the illustrated embodiment, implantable lead system 508 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), globus pallidus externus (GPe), medial forebrain bundle (MFB), periaquaductal gray (PAG), periventricular gray (PVG), habenula, subgenual cingulate cortex, ventral intermediate nucleus (VIM) of the thalamus, anterior nucleus (AN) or other nuclei of the thalamus, zona incerta, ventral capsule, ventral striatum, nucleus accumbens, and other white matter tracts connecting these and other structures. DBS is discussed as an example for the present subject matter, which can be applied to other brain stimulation (e.g., cortical stimulation) and other neurostimulation therapies.

Returning to FIG. 4, the IPG 404 can include a hermetically-sealed IPG case 422 to house the electronic circuitry of IPG 404. IPG 404 can include an electrode 426 formed on IPG case 422. In some embodiments, IPG case 422 can be used as electrode 426. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of stimulation device 104, including any of its examples discussed in this document, can include a control circuit used to control delivery of the neurostimulation energy (including stimulation programming circuit 320 and its various examples discussed in this document). The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

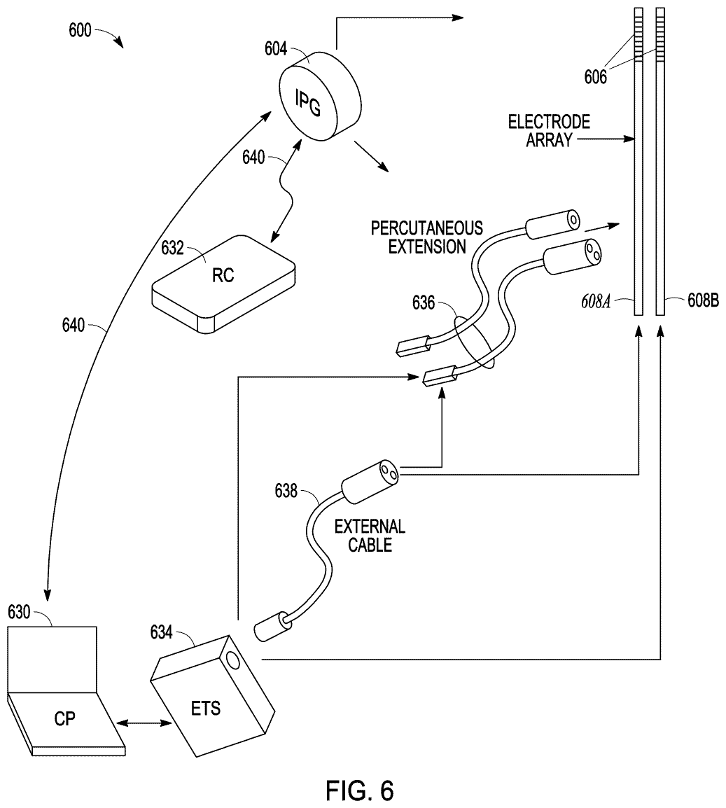

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS) 634. IPG 404 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an embodiment of system 100, with IPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604,

RC 632 also communicates with IPG 604 using a wireless link 340. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630, CP 630 may communicate with the RC 632 using a wired or wireless communications link. In some embodiments, CP 630 is able to program RC 632 when remotely located from RC 632.

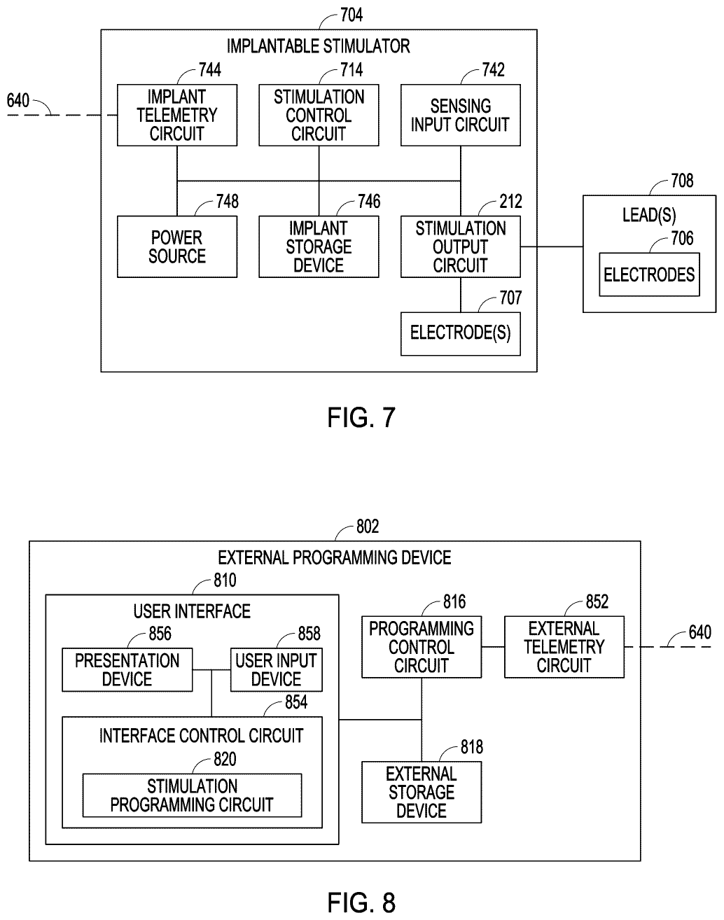

FIG. 7 illustrates an embodiment of implantable stimulator 704 and one or more leads 708 of an implantable neurostimulation system, such as implantable system 600. Implantable stimulator 704 represents an embodiment of stimulation device 104 or 204 and may be implemented, for example, as IPG 604. Lead(s) 708 represents an embodiment of lead system 208 and may be implemented, for example, as implantable leads 608A and 608B. Lead(s) 708 includes electrodes 706, which represents an embodiment of electrodes 106 or 206 and may be implemented as electrodes 606.

Implantable stimulator 704 may include a sensing input circuit (also known as a sensing circuit) 742 that provides the stimulator with a sensing capability, stimulation output circuit 212, a stimulation control circuit 714, an implant storage device 746, an implant telemetry circuit 744, a power source 748, and one or more electrodes 707. Sensing input circuit 742 senses one or more physiological signals for purposes of patient monitoring and/or feedback control of the neurostimulation. Examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707, and delivers each of the neurostimulation pulses through a set of electrodes selected from electrodes 706 and electrode(s) 707. Stimulation control circuit 714 represents an embodiment of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 stores values of the plurality of stimulation parameters. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640, and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing input circuit 742.

In various embodiments, sensing input circuit 742, stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an embodiment of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). For example, because DBS is often indicated for movement disorders which are assessed through patient activities, gait, balance, etc., allowing patient mobility during programming and assessment is useful. Therefore, when system 600 is intended for applications including DBS, wireless communication link 640 includes at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time (and allows the user to do so) to shape the stimulation like a piece of clay (pushing/pulling one spot at a time).

Programming control circuit 816 represents an embodiment of programming control circuit 316 and generates the plurality of stimulation parameters, which is to be transmitted to implantable stimulator 704, based on a specified stimulation configuration (e.g., the pattern of neurostimulation pulses as defined by one or more stimulation waveforms and one or more stimulation fields, or at least certain aspects of the pattern). The stimulation configuration may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an embodiment of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes a stimulation programming circuit 820.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. In some embodiments, one or more additional user interfaces (e.g., in one or more additional programming devices) are used in an online (e.g., real-time) programming mode and/or an offline (e.g., composing) programming mode. Programming control circuit 816 dynamically updates values of the plurality of stimulation parameters in response to changes in the set of one or more stimulation waveforms, and transmits the plurality of stimulation parameters with the updated values to implantable stimulator 704.

Stimulation control circuit 820 represents an example of stimulation programming circuit 320 and can be configured for determining the therapeutic effect threshold for the patient and determining stimulation parameters for the patient using the therapeutic effect threshold. For example, stimulation control circuit 820 can be used to programming implantable stimulator 704 for operating with external programming device 802 to sense a physiological signal from the patient while delivering neurostimulation to the patient for determining the therapeutic effect threshold for the patient. Stimulation control circuit 820 can then be used to determine stimulation parameters using the therapeutic effect threshold for programming implantable stimulator 704 to deliver a neurostimulation therapy to the patient according to the stimulation parameters. In various embodiments, User interface 810 can be configured to allow the user to participate in the programming process, such as by presenting the determined therapeutic effect threshold to the user and allowing the user to determining and/or accepting values of one or more stimulation parameters upon knowing the therapeutic effect threshold.

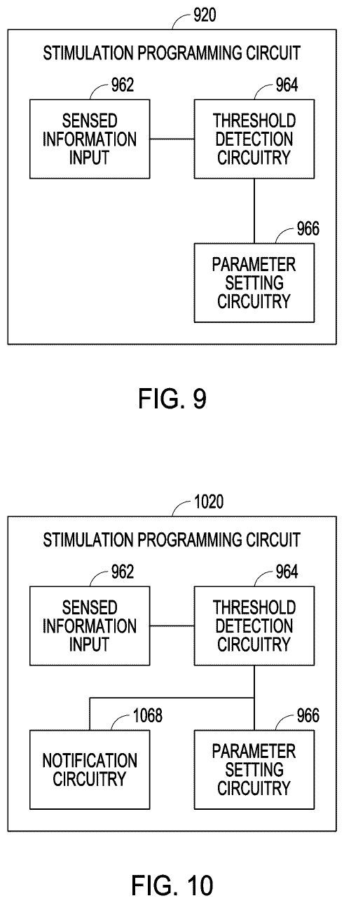

FIG. 9 illustrates an embodiment of a stimulation programming circuit 920 of a programming device of a neurostimulation system, such as programming device 302 or external programming device 802. Stimulation programming circuit 920 can represent an example of stimulation programming circuit 320 or 820 and can determine a therapeutic effect threshold for a patient and determine stimulation parameters for the patient based on the therapeutic effect threshold.

Stimulation programming circuit 920 can include a sensed information input 962, threshold detection circuity 964, and parameter setting circuity 966. Sensed information input 962 can receive sensed information including a physiological signal indicative of a therapeutic effect or information derived from the physiological signal and indicative of the therapeutic effect. The therapeutic effect includes an improvement of a condition being treated using neurostimulation. Threshold detection circuity 964 can determine a therapeutic effect threshold of an adjustable parameter of the set of stimulation parameters using the received sensed information. The therapeutic effect threshold is a minimum value of the adjustable parameter for the delivery of the neurostimulation according to the set of stimulation parameters to produce the therapeutic effect. The adjustable parameter can be any stimulation parameter having a value that can be programmed for the patient and/or adjusted (manually and/or automatically) for the patient. Parameter setting circuity 966 can determine the set of stimulation parameters for producing the therapeutic effect in the patient using the therapeutic effect threshold of the adjustable parameter. In various embodiments, the set of stimulation parameters can define a pattern of neurostimulation including one or more stimulation waveforms and one or more stimulation fields or a neurostimulation program including one or more patterns of neurostimulation. A programming control circuit such as programming control circuit 816 can generate information for programming a stimulation device to control the delivery of the neurostimulation according to the set of stimulation parameters. Examples of the stimulation device include stimulation device 104 and its various examples discussed above (e.g., stimulation device 204, IPG 404, IPG 504, IPG 604, and implantable stimulator 704).

In various embodiments, the sensed information received by sensed information input can include a physiological signal sensed from the patient, such as by using the stimulation device (e.g., using implantable stimulator 704, with sensing circuit 742). The physiological signal can be a neural signal indicative of a therapeutic effect, such as a neural signal including ERNA. In various embodiments, the sensed information received by sensed information input can include information derived from the physiological signal, such as a parameter that is measured from the physiological signal and indicative of the therapeutic effect. In one embodiment, the sensed information includes the physiological signal sensed in real time to allow for determination of the therapeutic effect threshold of the adjustable parameter by threshold detection circuity 964 in real time. In another embodiment, the sensed information includes historical and/or aggregate data, such as the physiological signal that is previously sensed and/or information derived from the previously sensed physiological signal, to allow for determination of the therapeutic effect threshold of the adjustable parameter by threshold detection circuity 964 used stored data.

In various embodiments, threshold detection circuity 964 can determine the therapeutic effect threshold of the adjustable parameter for a specified therapeutic effect, a particular patient, and/or a specified neurostimulation setting (e.g., a particular set of parameters defining one or more stimulation waveforms and one or more stimulation fields). An example of the adjustable parameter is a stimulation amplitude (e.g., a pulse amplitude), and the therapeutic effect threshold of the stimulation amplitude can be referred to as a minimum effective amplitude. In various embodiments, the adjustable parameter can be any stimulation parameter that can be adjusted for the therapeutic effect. Threshold detection circuity 964 can receive the physiological signal sensed while the neurostimulation is delivered to the patient with the value of the adjustable parameter swept across a test range, measure a biomarker parameter from the sensed physiological signal, compare the measured biomarker parameter to a sensed effect threshold, and identify the therapeutic effect threshold from the test range. The therapeutic effect threshold is a minimum value at which the measured biomarker parameter reaches or exceeds the sensed effect threshold. An example of the biomarker parameter is an amplitude measured from the neural signal (e.g., an ERNA amplitude measured from the neural signal including ERNA). The test range across with the adjustable parameter is swept can be determined, for example, for a comprehensive sweeping for determining a “global” therapeutic effect threshold (e.g., for a pattern of neurostimulation), a targeted sweeping for determine a “regional” therapeutic effect threshold (e.g., for a specific set of stimulation parameters used in a segment of the pattern of neurostimulation), or a set stimulation space (e.g., a set of electrodes known to be associated with a therapeutic effect).

In various embodiments, parameter setting circuity 966 can determine the set of stimulation parameters by executing a programming algorithm using the therapeutic effect threshold of the adjustable parameter. The programming algorithm can set one or more values and/or one or more value ranges for the adjustable parameter of the set of stimulation parameters. The one or more value ranges can each have a lower bound and an upper bound, with the lower bound being set based on the therapeutic effect threshold. A value range of a stimulation amplitude (e.g., pulse amplitude) can have the lower bound set to the minimum effective amplitude. The minimum effective amplitude can vary substantially across patients and stimulation settings, as the minimum therapeutic effect depends on the stimulation amplitude and the radius of stimulation (distance between the target and the electrode). Thus, parameter setting circuity 966 allows the therapeutic effect threshold, such as the minimum effective amplitude, to be determined for each stimulation setting with each patient. If a programming algorithm is prioritizing a setting in a region of stimulation, the programming algorithm can trigger a request for an updated determination of the therapeutic effect threshold (with different range, region, or regions of stimulation space) to assist in prioritization determination.

FIG. 10 illustrates an embodiment of a stimulation programming circuit 1020, which is a further embodiment of stimulation programming circuit 920 and can represent another example of stimulation programming circuit 320 or 820. Stimulation programming circuit 1020 can include sensed information input 962, threshold detection circuity 964, parameter setting circuity 966, and notification circuitry 1068. Notification circuitry 1068 can present notifications using a presentation device (such as presentation device 856) based on the therapeutic effect threshold of the adjustable parameter. In addition to using the therapeutic effect threshold of the adjustable parameter to set values of stimulation parameters, stimulation programming circuit 1020 can use the therapeutic effect threshold of the adjustable parameter to indicate changes in the neurostimulation system that can affect the therapeutic effect in the patient. For example, notification circuitry 1068 can set a flag to indicate to the user that a change has occurred in the neurostimulation system, particularly when the change is of a type that can affect the efficacy of the neurostimulation.

In various embodiments, threshold detection circuitry 964 can determine the therapeutic effect threshold of the adjustable parameter over time, and notification circuitry 1068 can monitor the therapeutic effect threshold determined over time and detect each substantial change of the therapeutic effect threshold. For example, threshold detection circuitry 964 can detect a substantial change of the therapeutic effect threshold when the difference between a currently determined therapeutic effect threshold and a previously determined therapeutic effect threshold exceeds a threshold. In response to a detection of the substantial change, notification circuitry 1068 can generate a notification (e.g., a flag or an alarm) for the user (e.g., to react to an issue with the neurostimulation system or the patient that cannot be automatically addressed) and/or for the neurostimulation system (e.g., to address an issue by automatically adjusting system settings).

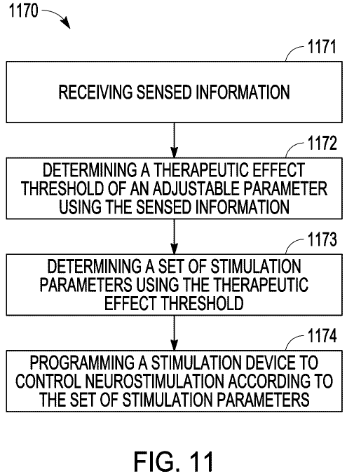

FIG. 11 illustrates an embodiment of a method 1170 for setting stimulation parameters for delivering neurostimulation to a patient using a therapeutic effect threshold. Method 1170 can be performed using a neurostimulation system such as system 100, including the various examples of the system and its components discussed in this document. For example, stimulation programming circuit 920 or 1020 can be included in the neurostimulation system (e.g., a system with implantable stimulator 704 and external programming device 802) and configured (e.g., programmed) for performing method 1170 using that neurostimulation system.

At 1171, sensed information is received. The sensed information can include a physiological signal indicative of a therapeutic effect improving a condition of the patient being treated using the neurostimulation and/or information (e.g., one or more parameters) derived from the physiological signal.

At 1172, a therapeutic effect threshold of an adjustable parameter of a set of stimulation parameters is determined using the sensed information. The adjustable parameter can include any stimulation parameter whose value can be set and adjusted for efficacy of the neurostimulation (i.e., effectiveness in producing the therapeutic effect). The therapeutic effect threshold is a minimum value of the adjustable parameter for the delivery of the neurostimulation according to the set of stimulation parameters to produce the therapeutic effect.

At 1173, the set of stimulation parameters is determined for producing the therapeutic effect using the therapeutic effect threshold of the adjustable parameter. At 1174, a stimulation device (e.g., implantable stimulator 704) is programmed to control the delivery of the neurostimulation according to the set of stimulation parameters.

Method 1170 can be performed in real time, with the physiological signal being sensed and received in real time at 1170. When real-time sensed information is not available, the therapeutic effect threshold of the adjustable parameter can be determined (e.g., estimated) using historical and/or aggregated data.

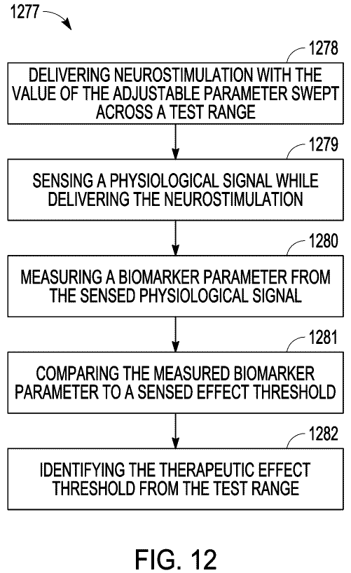

FIG. 12 illustrates an embodiment of a method 1277 for determining the therapeutic effect threshold. Method 1277 can represent an example for performing steps 1171 and 1172 of method 1170 using the neurostimulation system.

At 1278, neurostimulation is delivered to the patient with the value of the adjustable parameter swept across a test range. The test range can be determined based on the purpose of the sweeping (e.g., for determining the adjustable parameter for a complete neurostimulation program or for a particular segment of the program).

At 1279, the physiological signal indicative of the therapeutic effect improving the condition of the patient is sensed. An example of the physiological signal is a neural signal including ERNA.

At 1280, a biomarker parameter is measured from the physiological signal sensed while the neurostimulation is delivered at 1278. An example of the biomarker parameter is an amplitude of the ERNA.

At 1281, the measured biomarker parameter is compared to a sensed effect threshold. The sensed effect threshold is the value of the biomarker parameter corresponding the minimum therapeutic effect detected by the neurostimulation system or a person (e.g., the user and/or the patient).

At 1282, the therapeutic effect threshold is identified from the test range. The therapeutic effect threshold is a minimum value at which the measured biomarker parameter reaches or exceeds the sensed effect threshold.

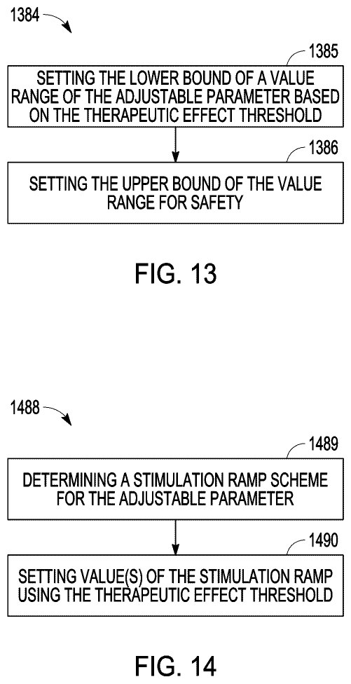

FIG. 13 illustrates an embodiment of a method 1384 for setting a value range for the adjustable parameter using the therapeutic effect threshold. Method 1384 can represent an example for performing part of step 1174 of method 1170 using the neurostimulation system. The value range for the adjustable parameter can have an upper bound and a lower bound. In one example, the value range is determined for identifying an optimal value of the adjustable parameter in a stimulation setting for the patient.

At 1385, the lower bound of the value range of the adjustable parameter is set based on the therapeutic effect threshold of the adjustable parameter. In various embodiments, the low bound can be automatically set to the therapeutic effect threshold or to the therapeutic effect threshold multiplied by a factor. In various other embodiments, the therapeutic effect threshold can be presented with the value range to the user using a user interface of the neurostimulation system (e.g., user interface 810), and the user is allowed to set the lower bound using the user interface.

At 1386, the upper bound of the value range of the adjustable parameter is set for safety, such as based on a side effect of the neurostimulation in the patient. In various embodiments, the upper bound can be set by querying the user using the user interface. The user can determine one or more side effects, for example based on the patient's perceivable response to the neurostimulation and/or statistical data collected from a patient population or simulations). In various embodiments, the upper bound can be set automatically based on a known or estimated location of a side effect in the patient.

FIG. 14 illustrates an embodiment of a method 1488 for setting a stimulation ramp for the adjustable parameter using the therapeutic effect threshold. Method 1384 can represent an example for performing part of step 1174 of method 1170 using the neurostimulation system. In various embodiments, the user can apply a stimulation ramp, e.g., ramping up the value of the adjustable parameter to a target value over a period such as hours, days, or weeks. In one embodiment, the stimulation ramp includes a value range set in a manner identical or substantially similar to that of method 1384.

At 1489, a stimulation ramp scheme is determined for the adjustable parameter. Examples of the stimulation ramp scheme include: (1) ramping up the value of the adjustable parameter (i.e., increase the value of the adjustable parameter at specified increments) over a specified period, (2) ramping up the value of the adjustable parameter at a first rate until the therapeutic effect threshold is reached and at a second rate after the therapeutic effect threshold is reached, (3) ramping up the value of the adjustable parameter until the therapeutic effect threshold is reached, and (4) ramping up the value of the adjustable parameter until the therapeutic effect threshold is reached and then to a target value determined using the therapeutic effect threshold.

At 1490, one or more values (the lower bound and/or a turning point) of the stimulation ramp are set using the therapeutic effect threshold. For example, the lower bound or the turning point can be set to the therapeutic effect threshold.

In some embodiments, in which the patient is receiving pharmaceutical and neurostimulation therapies, as medications are adjusted, the therapeutic effect threshold is updated, for example by repeating method 1277. For example, the minimum effective amplitude of a stimulation amplitude can be reevaluated and updated following each adjustment in medication (e.g., 10 mg drug with the lower bound of stimulation amplitude set to 2 mA for week 1, and 20 mg drug with the lower bound of stimulation amplitude set to 1.5 mA for week 2).

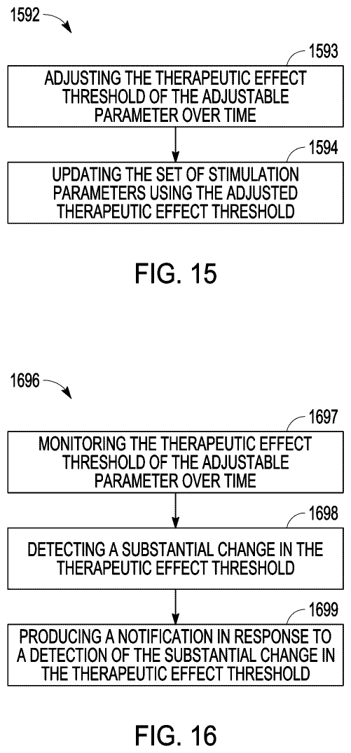

FIG. 15 illustrates an embodiment of a method 1592 for setting the stimulation parameters using the therapeutic effect threshold adjusted over time. Method 1592 can represent an example for repeating method 1170 according to a schedule or as needed using the neurostimulation system. The patient's conditions may change as the neurostimulation is applied and/or medications wash in or out, resulting in changes of the therapeutic effect threshold over time.

At 1593, the therapeutic effect threshold of the adjustable parameter is adjusted over time, for example in response to changes in the patient's response to the neurostimulation. In various embodiments, steps 1171, 1172, and 1173 of method 1170 can be repeated when being triggered. Example of the trigger can be a schedule, such as on a periodic basis (e.g., hours, minutes) and/or a request command (e.g., issued automatically by the neurostimulation system or by the user using the user interface) that can result from reduced effectiveness of the neurostimulation.

At 1594, the set of stimulation parameters is updated using the adjusted therapeutic effect threshold. In various embodiments, step 1174 of method 1170 can be repeated in response to each change in the therapeutic effect threshold of the adjustable parameter. This ensures effectiveness of the neurostimulation when the patient's conditions change.

FIG. 16 illustrates an embodiment of a method 1696 for producing a notification based on a substantial change in the therapeutic effect threshold. Method 1696 can be performed using the same neurostimulation system used to perform method 1170 and performed as the performance of method 1170 is repeated to update the therapeutic effect threshold over time. For example, stimulation programming circuit 1020 can be included in the neurostimulation system, with notification circuitry 1068 configured (e.g., programmed) to perform method 1696.

At 1697, the therapeutic effect threshold of the adjustable parameter is monitored over time, for example by tracking the result of each performance of method 1592. At 1698, a substantial change in the therapeutic effect threshold is detected. The substantial change can be a change of the therapeutic effect threshold from a previously determined value to a currently determined value that reaches or exceeds a specified margin.

At 1699, a notification is produced in response to a detection of the substantial change in the therapeutic effect threshold. The notification can be presented using the user interface (e.g., user interface 810) or transmitted to another device to attract the user's attention. In various embodiments, the notification can indicate a change in the neurostimulation system that affect the efficacy and/or safety of the neurostimulation, or other unanticipated change that substantially affects the therapeutic effect threshold. Automatic responses by the neurostimulation system and/or manual responses by the user can be triggered by the notification.

In various embodiments, a non-transitory computer-readable storage medium can include instructions, which when executed by a neurostimulation system (e.g., the system with implantable stimulator 704 and external programming device 802, as discussed above with references to FIGS. 11-16), cause the neurostimulation system to perform methods 1170, 1277, 1384, 1488, 1592, and/or 1696. For example, when the system with implantable stimulator 704 and external programming device 802 is configured (e.g., programmed) for performing any one or any combination of methods 1170, 1277, 1384, 1488, 1592, and/or 1696, external storage device 818 can store the instructions as the non-transitory computer-readable storage medium. A portable storage medium, or a storage device in a computer or other device, can also be used to store the instructions as the non-transitory computer-readable storage medium, such as for programming stimulation programming circuit 820 to function as stimulation programming circuit 920 or 1020.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to a patient using a stimulation device, the system comprising:
a programming control circuit configured to generate information for programming the stimulation device to control the delivery of the neurostimulation according to a set of stimulation parameters; and
a stimulation programming circuit configured to determine the set of stimulation parameters, the stimulation programming circuit including:
a sensed signal input configured to receive sensed information indicative of a therapeutic effect improving a condition being treated using the neurostimulation;
threshold detection circuity configured to determine a therapeutic effect threshold of an adjustable parameter of the set of stimulation parameters using the sensed information, the therapeutic effect threshold being a minimum value of the adjustable parameter for the delivery of the neurostimulation according to the set of stimulation parameters to produce the therapeutic effect; and
parameter setting circuity configured to determine the set of stimulation parameters for producing the therapeutic effect using the therapeutic effect threshold of the adjustable parameter.

2. The system of claim 1, wherein the parameter setting circuity is configured to determine the set of stimulation parameters for producing the therapeutic effect in the patient using a minimum effective amplitude of a stimulation amplitude of the set of stimulation parameters, and the threshold detection circuity is configured to determine the minimum effective amplitude using the sensed information, the minimum effective amplitude being a minimum value of the stimulation amplitude for the delivery of the neurostimulation according to the set of stimulation parameters to produce the therapeutic effect in the patient.

3. The system of claim 1, wherein the threshold detection circuity is configured to determine the therapeutic effect threshold of the adjustable parameter using a physiological signal of the sensed information, the physiological signal sensed while the neurostimulation is delivered to the patient with the value of the adjustable parameter swept across a test range.

4. The system of claim 3, wherein the threshold detection circuity is configured to:
measure a biomarker parameter from the physiological signal;
compare the measured biomarker parameter to a sensed effect threshold; and
identify the therapeutic effect threshold of the adjustable parameter from the test range, the therapeutic effect threshold being a minimum value at which the measured biomarker parameter reaches or exceeds the sensed effect threshold.

5. The system of claim 1, wherein the set of stimulation parameters defines a pattern of neurostimulation including one or more stimulation waveforms and one or more stimulation fields, and the sensed signal input is configured to receive a neural signal including evoked resonant neural activity (ERNA).

6. The system of claim 1, wherein the parameter setting circuity is configured to set a value range of the adjustable parameter having a lower bound and an upper bound, the lower bound set based on the therapeutic effect threshold of the adjustable parameter.

7. The system of claim 6, wherein the parameter setting circuity is configured to set the value range of the adjustable parameter for identifying an optimal value of the adjustable parameter in a stimulation setting for the patient and to set the lower bound automatically to the therapeutic effect threshold of the adjustable parameter multiplied by a factor.

8. The system of claim 6, further comprising a user interface including a presentation device and a user input device, and wherein the parameter setting circuity is configured to set the value range of the adjustable parameter for identifying an optimal value of the adjustable parameter in a stimulation setting for the patient, to present the therapeutic effect threshold of the adjustable parameter using the presentation device, and to allow the lower bound to be set using the user input device.

9. The system of claim 8, wherein the stimulation programming circuit further comprises a notification circuitry to detect a substantial change in the therapeutic effect threshold of the adjustable parameter and to present a notification using the presentation device in response to a detection of the substantial change.

10. The system of claim 1, wherein the parameter setting circuity is configured to determine values of the adjustable parameter in a stimulation program including a stimulation ramp during which the value of the adjustable parameter changes in increments from or to a bound or turning point and to set the bound or turning point based on the therapeutic effect threshold of the adjustable parameter.

11. A method for delivering neurostimulation to a patient using a stimulation device, the method comprising:
receiving sensed information indicative of a therapeutic effect improving a condition being treated using the neurostimulation;
determining a therapeutic effect threshold of an adjustable parameter of a set of stimulation parameters using the sensed information, the therapeutic effect threshold being a minimum value of the adjustable parameter for the delivery of the neurostimulation according to the set of stimulation parameters to produce the therapeutic effect;
determining the set of stimulation parameters for producing the therapeutic effect using the therapeutic effect threshold of the adjustable parameter; and
programming the stimulation device to control the delivery of the neurostimulation according to the set of stimulation parameters.

12. The method of claim 11, further comprising receiving stored sensed information including at least one of a previously sensed physiological signal or information derived from the previously sensed physiological signal, and wherein determining the therapeutic effect threshold of the adjustable parameter comprises determining the therapeutic effect threshold of the adjustable parameter using the received stored sensed information.

13. The method of claim 11, further comprising sensing a physiological signal from the patient in real time, and wherein receiving the sensed information comprises receiving the physiological signal in real time, and determining the therapeutic effect threshold of the adjustable parameter comprises determining the therapeutic effect threshold of the adjustable parameter in real time.

14. The method of claim 13, wherein the physiological signal comprises a neural signal including evoked resonant neural activity (ERNA).

15. The method of claim 13, wherein determining the therapeutic effect threshold of the adjustable parameter comprises:
measuring a biomarker parameter from the physiological signal sensed while delivering the neurostimulation to the patient with the value of the adjustable parameter swept across a test range;
comparing the measured biomarker parameter to a sensed effect threshold; and
identifying the therapeutic effect threshold of the adjustable parameter from the test range, the therapeutic effect threshold being a minimum value at which the measured biomarker parameter reaches or exceeds the sensed effect threshold.

16. The method of claim 11, wherein determining the set of stimulation parameters comprises determining a value range of the adjustable parameter having a lower bound and an upper bound, including setting the lower bound based on the therapeutic effect threshold of the adjustable parameter.

17. The method of claim 16, wherein setting the lower bound based on the therapeutic effect threshold of the adjustable parameter comprises:
presenting the therapeutic effect threshold using a user interface; and
allowing a user to set the lower bound using the user interface.

18. The method of claim 11, wherein determining the set of stimulation parameters comprises:
determining values of the adjustable parameter in a stimulation program including a stimulation ramp during which the value of the adjustable parameter changes in increments from or to a bound or turning point; and
setting the bound or turning point based on the therapeutic effect threshold of the adjustable parameter.

19. The method of claim 11, further comprising:
monitoring the therapeutic effect threshold of the adjustable parameter over time;
detecting an unanticipated significant change of the therapeutic effect threshold of the adjustable parameter; and
producing a notification in response to a detection of the unanticipated significant change.

20. A non-transitory computer-readable storage medium including instructions, which when executed by a system, cause the system to perform a method for delivering neurostimulation to a patient using a stimulation device, the method comprising:
receiving sensed information indicative of a therapeutic effect improving a condition being treated using the neurostimulation;
determining a therapeutic effect threshold of an adjustable parameter of a set of stimulation parameters using the sensed information, the therapeutic effect threshold being a minimum value of the adjustable parameter for the delivery of the neurostimulation according to the set of stimulation parameters to produce the therapeutic effect;
determining the set of stimulation parameters for producing the therapeutic effect using the therapeutic effect threshold of the adjustable parameter; and
programming the stimulation device to control the delivery of the neurostimulation according to the set of stimulation parameters.
